(19) United States
(12) Patent Application Publication
(10) Pub. No.: US 2007/0254019 A1
(43) Pub. Date: Nov. 1, 2007

(54) METHOD FOR TREATING BRAIN CANCER

(76) Inventors: William Zamboni, Pittsburgh, PA (US); Charles Engbers, Aptos, CA (US); Ning Yu, Saratoga, CA (US); Margaret Tonda, Santa Clara, CA (US); Barbara Stewart, Palo Alto, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/686,326

(22) Filed: Mar. 14, 2007

Related U.S. Application Data
(60) Provisional application No. 60/782,751, filed on Mar. 15, 2006.
(60) Provisional application No. 60/870,714, filed on Dec. 19, 2006.

(51) Int. Cl. A61K 31/4355; A61K 9/127; A61P 35/00; A61P 35/04

(52) U.S. Cl. 424/450; 514/283

(57) ABSTRACT

A method for treating brain cancer is described. The method involves administering a chemotherapeutic agent, such as a topoisomerase inhibitor, entrapped in liposomes.

### CROSS REFERENCE TO RELATED U.S. APPLICATION DATA

The present application claims the benefit of provisional application 60/782,751, filed Mar. 15, 2006 and 60/870,714, filed Dec. 19, 2006 entitled Method for Treating Brain Cancer, of which is incorporated by reference in its entirety.

### TECHNICAL FIELD

A method of treating brain cancer is provided. More specifically, a method of treating brain cancer with liposome-entrapped topoisomerase inhibitors is provided.

### BACKGROUND

It is currently unclear why within a patient with solid tumors there can be a reduction in the size of some tumors while other tumors can progress during or after treatment, even though the genetic composition of the tumors is similar (Jain, R K. Delivery of molecular medicine to solid tumors. Science, 271(5252);1079-1080:1996b; Zamboni et al., Relationship between tumor extracellular fluid exposure to topotecan and tumor response in human neuroblastoma xenograft and cell lines. Cancer Chemother Pharmacol 43 (4); 269-276:1999a; Balch et al., Cutaneous Melanoma. In Cancer: Principles and Practice in Oncology, 5th Ed, Devita V T, Hellman S, and Rosenberg S A, eds. Lippincott-Raven. 1947; 2006). Such variable antitumor responses within a single patient may be associated with inherent differences in tumor vascularity, capillary permeability, and/or tumor interstitial pressure that result in variable delivery of anticancer agents to different tumor sites. However, studies evaluating the intratumoral concentration of anticancer agents and factors affecting tumor exposure in preclinical models and patients are rare. It is logistically difficult to perform the extensive studies required to evaluate the tumor disposition of anticancer agents and factors that determine the disposition in patients with solid tumors, especially in tumors which are not easily accessible (Blochl-Daum et al., Measurement of extracellular fluid carboplatin kinetics in melanoma metastases with microdialysis. Br J Cancer 73(7); 920-924:1996; Muller et al., 5-fluorouracil kinetics in the interstitial tumor space: clinical response in breast cancer patients. Cancer Res 57 (13); 2598-2601:1997). Thus, there is an impending need to develop and implement techniques and methodologies to evaluate the disposition and exposure of anticancer agents within the tumor matrix.

Brain cancer is a disease that affects many individuals. Each year over 190,000 people in the United States and 10,000 people in Canada are diagnosed with a primary or metastatic brain tumor. Brain tumors are a leading cause of death from childhood cancer, accounting for almost a quarter of cancer deaths in children up to 19 years of age. Brain tumors are the second leading cause of cancer death in young adults ages 20-39. In view of the deadly nature of brain cancer, it would be beneficial if techniques and methodologies were available to evaluate the disposition and exposure of anticancer agents within brain tumor matrix so that an effective anticancer agent could be identified.

There are two types of brain cancers: primary brain tumors that originate in the brain and metastatic (secondary) brain tumors that originate from cancer cells that have migrated from other parts of the body. Primary brain cancer rarely spreads beyond the central nervous system, and death results from uncontrolled tumor growth within the limited space of the skull. Metastatic brain cancer indicates advanced disease and has a poor prognosis.

Primary brain tumors can be cancerous or noncancerous. Both types take up space in the brain and may cause serious symptoms (e.g., vision or hearing loss) and complications (e.g., stroke). All cancerous brain tumors are life threatening (malignant) because they have an aggressive and invasive nature. A noncancerous primary brain tumor is life threatening when it compromises vital structures (e.g., an artery).

Primary brain tumors are categorized by the type of tissue in which they first develop. The most common brain tumors are called glioma; they originate in the glial (supportive) tissue. There are a number of different types of gliomas:

Astrocytomas develop from small, star-shaped cells called astrocytes. They may arise anywhere in the brain or spinal cord. In adults, astrocytomas most often occur in the cerebrum, which is the largest part of the brain. The cerebrum fills most of the upper skull, and uses sensory information to tell us what is going on around us and tells our body how to respond. The left hemisphere controls the muscles on the right side of the body, while the right hemisphere controls the muscles on the left. The cerebrum also controls speech and emotions, as well as reading, thinking, and learning.

Brain stem gliomas arise in the brain stem, which controls many vital functions such as body temperature, blood pressure, breathing, hunger and thirst. The brain stem connects the brain with the spinal cord. Tumors in this area generally cannot be removed. Most brain stem gliomas are high-grade astrocytomas.

Ependymomas usually occur in the lining of the ventricles, or in the spinal cord. Although ependymomas can develop at any age, they most commonly arise in children and adolescents.

Oligodendrogliomas develop in the cells that produce myelin, the fatty covering that protects nerves. These tumors are very rare, and usually occur in the cerebrum. They are slow growing and generally do not spread into surrounding brain tissue. While they occur most often in middle-aged adults, they have been found in people of all ages.

There are other types of brain tumors that do not begin in glial tissue. Some of the most common are:

Meningiomas grow from the meninges, which are three thin membranes that surround the brain. These of tumors are usually benign. Because they grow very slowly, the brain may be able to adjust to their presence. Meningiomas frequently grow quite large before they cause symptoms. They occur most often in women ages 30 to 50.

Craniopharyngiomas develop in the area of the pituitary gland (the main endocrine gland, which produces hormones that control other glands and many body functions, especially growth) near the hypothalamus. These tumors are usually benign; however, they may sometimes be considered malignant because they may create pressure on, or damage the hypothalamus and affect vital functions such as body temperature, hunger, and thirst. These tumors occur most often in children and adolescents.

Germ cell tumors arise from developing sex (egg or sperm) cells, also known as germ cells. The most common type of germ cell tumor in the brain is the germinoma. Germinomas can form in the ovaries, testicles, chest, abdomen, as well as the brain.

Pineal region tumors occur in or around the pineal gland, a very small organ located in the center of the brain. The pineal gland produces melatonin, a hormone that plays an important role in the sleep-wake cycle. These tumors can be slow growing (pineocytoma), or fast growing (pineoblastoma). The pineal region is very difficult to reach, and these tumors often cannot be removed.

Several strategies have been explored to improve outcomes and prognosis of patients with brain cancer. Treatment for brain cancer depends on the age of the patient, the stage of the disease, the type and location of the tumor, and whether the cancer is a primary tumor or metastatic. The treatment plan is developed by an oncology team and the patient, but typically involves any combination of surgery, radiation, and chemotherapy.

Chemotherapy is generally preferred to surgery and radiation, but an effective chemotherapy treatment has not been identified. One of the problems with current chemotherapy approaches is that it is extremely difficult to achieve therapeutically effective levels of a chemical agent in brain tissue and brain tumors. Thus, there remains a need for the therapeutically effective delivery of a pharmaceutically therapeutic agent to brain tissue and/or brain tumors.

### SUMMARY

In one aspect, a method of treating brain cancer in a subject is provided by administering a liposome-entrapped topoisomerase inhibitor.

In another aspect, a method of treating brain cancer in a subject is provided by administering a lipsome-entrapped topoisomerase inhibitor, wherein the liposome has an outer surface coating of hydrophilic polymer chains.

In another aspect, a method of treating brain cancer in a subject is provided by administering a liposome-entrapped camptothecin or camptothecin derivative.

In another aspect, a method of treating brain cancer in a subject is provided by administering a liposome-entrapped CKD-602.

In addition to the exemplary aspects and embodiments described above, further aspects and embodiments will become apparent by reference to the drawings and by study of the following descriptions.

### BRIEF DESCRIPTION OF THE DRAWINGS

FIG. 1. Concentration versus time profiles of CKD-602 in plasma, tumor, and tissues after administration of non-liposomal CKD602. Samples were obtained after administration of vehicle, and at 5 min, 0.25, 0.5,1, 2, 4, 7,16, and 24 h after administration. Each time point represents the mean of three mice.

FIG. 2. Concentration versus time profiles of sum total CKD-602 in plasma, tumor, and tissues after administration of S-CKD602. Samples were obtained after administration of vehicle, and at 5 min, 0.25, 0.5, 1, 2, 4, 7,16, 24, 48, and 72 h after administration. Each time point represents the mean of three mice.

FIG. 3. Concentration versus time profile of CKD-602 in plasma, tumor, and tumor ECF after administration of non-liposomal CKD602. The plasma and tumor sum total concentration represent the mean of 3 mice at each time point. Microdialysis studies (n=3 to 4 mice per interval) were obtained every 20 min from 0 to 2 h and every 30 min from 4 to 8 h and 20 to 24 h after non-liposomal CKD602. The mean concentration in tumor ECF at each time point is represented by the open diamonds. The average concentration in the tumor ECF at each interval is represented by the solid diamonds and is connected by a dashed line.

FIG. 4. Concentration versus time profiles of CKD-602 in plasma, tumor, and tumor ECF after S-CKD602. Plasma and tumor sum total concentration represents the mean of 3 mice at each time point. Plasma profiles consists of sum total, encapsulated, and released CKD-602. Microdialysis studies (n=3 to 4 mice per interval) were obtained every 20 min from 0 to 2 h and every 30 min from 24 to 27 h, 48 to 51 h, and 72 to 75 h. The mean concentration in tumor ECF at each time point is represented by the solid diamonds and is connected by a dashed line.

### DETAILED DESCRIPTION

In one aspect, a method of treating brain cancer in a subject is provided by administering a liposome-entrapped topoisomerase inhibitor. As used herein, the term “subject” should be interpreted broadly and includes mammals in one embodiment, humans in another embodiment, and humans or patients in need of treatment in another embodiment.

Exemplary liposome-entrapped topoisomerase inhibitors are described in U.S. Pat. Nos. 6,355,268 and 6,465,008, which are incorporated herein by reference in their entirety. Specifically, but not exclusively, incorporated herein by reference is the description of a method for preparing liposomes containing a topoisomerase inhibitor, and the materials used in preparation of liposomes. Preparation of liposomes and selection of materials for preparing liposomes, is well known in the art, as exemplified in U.S. Pat. Nos. 6,355,268 and 6,465,008. While the literature discloses the use of liposomes containing a topoisomerase inhibitor to treat tumors, and specifically human colon tumors, it appears from a review of the foregoing that liposomes entrapped with a topoisomerase inhibitor have not been reported for the treatment of brain tumors, and specifically human brain tumors.

In another aspect, a method of treating brain cancer in a subject is provided by administering a liposome-entrapped topoisomerase inhibitor that is camptothecin or a camptothecin derivative. For example, the camptothecin derivative can be 9-aminocamptothecin, 7-ethylcamptothecin, 10-hydroxycamptothecin, 9-nitrocamptothecin, 10,11-methlyenedioxycamptothecin, 9-amino-10,11-methylenedioxycamptothecin or 9-chloro-10,11-methylenedioxycamptothecin, irinotecan, topotecan, (7-(4-methylpiperazinomethylene)-10,11-ethylenedioxy-20(S)-camptothecin, 7-(4-methylpiperazinomethylene)-10,11-methylenedioxy-20(S)-camptothecin. The topoisomerase inhibitor can also be a topoisomerase I/II inhibitor, such as 6-[[2-(dimethylamino)-ethyl]amino]-3-hydroxy-7H-indeno[2,1-c]quinolin-7-on e dihydrochloride, azotoxin or 3-methoxy-11H-pyrido[3′,4′-4,5]pyrrolo[3,2-c]quinoline-1,4-dione.

In one embodiment, the liposome-entrapped topoisomerase inhibitor excludes liposome-entrapped doxorubicin. In another embodiment, the liposome-entrapped topoisomerase inhibitor excludes liposome-entrapped topoisomerase inhibitor II compounds, such as doxorubicin. It will be appreciated that a topoisomerase inhibitor II compound is one that inhibits or reduces the action of topoisomerase II enzyme. A topoisomerase inhibitor I compound is one that inhibits or reduces the action of topoisomerase I enzyme. A topoisomerase I/II inhibitor refers to any compound that inhibits or reduces the action of both topoisomerase I enzyme and topoisomerase II enzyme.

In another aspect, a method of treating brain cancer in a subject is provided by administering a liposome-entrapped topoisomerase inhibitor, wherein the inhibitor is selected from the group consisting of the camptothecin analogues, topotecan, MPE-camptothecin and CKD-602. In a further aspect, a method of treating brain cancer in a subject by administering a liposome-entrapped topoisomerase inhibitor, wherein the inhibitor is CKD-602. CKD-602, a camptothecin analogue, inhibits topoisomerase I which prevents DNA replication and causes apoptosis.

U.S. Pat. Nos. 6,355,268 and 6,465,008 discloses a liposome entrapped with the topoisomerase inhibitor CKD-602. The liposome is comprised of phospholipid covalently bound to methoxypolyethylene glycol and entrapped with CKD-602 (any of the foregoing liposome-entrapped topoisomerase inhibitor formulations can have an outer surface coating of hydrophillic polymer chains such as, but not limited to, methoxypolyethylene glycol or polyethylene glycol or polyethylene glycol having a molecular weight between 500-5,000 daltons). Non-liposomal CKD602 is approved in South Korea in relapsed ovarian cancer and as a first line agent in small cell lung cancer. Once in the tumor, the liposomes are localized in the extracellular fluid (ECF) surrounding the tumor cell, but do not enter the cell (Harrington et al., Phase I-II study of pegylated liposomal cisplatin (SPI-077) in subjects with inoperable head and neck cancer. Ann Oncol 12 (4);493-496:2001 a; Harrington et al., Effective targeting of solid tumors in subjects with locally advanced cancers by radiolabeled pegylated liposomes. Clin Cancer Res 7 (2); 243-254:2001 b). Thus, for the liposomes to deliver the active form of the anticancer agent, such as doxorubicin in the case of DOXIL, the drug must be released from the liposome into the tumor ECF and then diffuse into the cell (Zamboni W C. Use of microdialysis in preclinical and clinical development. In: Handbook of Pharmacokinetics and Pharmacodynamics of Anti-Cancer Drugs, 1^{st} Ed, Figg W D, McLeod H, eds. Humana Press. 2004; Zamboni et al., Systemic and tumor disposition of platinum after administration of cisplatin or STEALTH liposomal-cisplatin formulations (SPI-077 and SPI-077 B103) in a preclinical tumor model of melanoma. Cancer Chemother Pharmacol 53 (4); 329-336:2004). As a result, the ability of the liposome to carry the anticancer agent to the tumor and release it into the ECF are equally important factors in determining the antitumor effect of liposomal encapsulated anticancer agents. The cytotoxicity of camptothecin analogues is related to the duration of exposure at or above the therapeutic threshold.

In another aspect, a method of treating brain cancer in a subject is provided by administering a liposome-entrapped topoisomerase inhibitor, wherein the method treats primary brain cancer.

In another aspect, a method of treating brain cancer in a subject is provided by administering a liposome-entrapped topoisomerase inhibitor, wherein the method treats secondary brain cancer.

In another aspect, a method of treating brain cancer in a subject is provided by administering a liposome-entrapped topoisomerase inhibitor, wherein the method treats glioma brain cancer.

In another aspect, a method of treating brain cancer in a subject is provided by administering a liposome-entrapped topoisomerase inhibitor, wherein the method treats non-glioma brain cancer.

In another aspect, a method of treating brain cancer in a subject is provided by administering a liposome-entrapped topoisomerase inhibitor, wherein the liposome is composed of a vesicle-forming lipid and between about 1-20 mole percent of a vesicle-forming lipid derivatised with a hydrophilic polymer, said liposomes being formed under conditions that distribute the polymer on both sides of the liposomes' bilayer membranes.

In another aspect, a method of treating brain cancer in a subject is provided by administering a liposome-entrapped topoisomerase inhibitor, wherein the liposome is composed of a vesicle-forming lipid and between about 1-20 mole percent of a vesicle-forming lipid derivatised with a hydrophilic polymer, said liposomes being formed under conditions that distribute the polymer on both sides of the liposomes' bilayer membranes, wherein the hydrophilic polymer is polyethyleneglycol having a molecular weight between 500 and 5,000 daltons and the vesicle-forming lipid is selected from the group consisting of hydrogenated soy phosphatidylcholine, distearoylphosphatidylcholine and sphingomyelin.

In another aspect, a method of treating brain cancer in a subject is provided by administering a liposome-entrapped topoisomerase inhibitor, wherein the liposome is composed of a vesicle-forming lipid and between about 1-20 mole percent of a vesicle-forming lipid derivatised with a hydrophilic polymer, said liposomes being formed under conditions that distribute the polymer on both sides of the liposomes' bilayer membranes, wherein the vesicle-forming lipid is selected from the group consisting of hydrogenated soy phosphatidylcholine, distearoylphosphatidylcholine and sphingomyelin.

In another aspect, a method of treating brain cancer in a subject is provided by administering a liposome-entrapped topoisomerase inhibitor, wherein the topoisomerase inhibitor is entrapped in the liposomes at a concentration of at least about 0.10 μM drug per μM lipid.

In another aspect, a method of treating brain cancer in a subject is provided by administering a liposome-entrapped topoisomerase inhibitor, wherein the liposomes have an inside/outside ion gradient sufficient to retain the topoisomerase inhibitor within the liposomes at the specified concentration prior to in vivo administration, and wherein said liposome-entrapped topoisomerase inhibitor has a longer blood circulation lifetime than the topoisomerase inhibitor in free form.

In another aspect, a method of treating brain cancer in a subject is provided by administering a liposome-entrapped topoisomerase inhibitor, wherein the liposomes include a vesicle-forming lipid having a phase transition temperature above 37° C.

In another aspect, a method of treating brain cancer in a subject is provided by administering a liposome-entrapped topoisomerase inhibitor, wherein the liposomes are composed of 20-94 mole percent hydrogenated soy phosphatidylcholine, 1-20 mole percent distearoyl phosphatidylethanolamine derivatized with polyethyleneglycol and 5-60 mole percent cholesterol; or 30-65 mole percent hydrogenated soy phosphatidylcholine; 5-20 mole percent distearoyl phosphatidylethanolamine derivatized with polyethyleneglycol, and 30-50 mole percent cholesterol; or 20-94 mole percent distearoyl phosphatidylcholine and 1-20 mole percent distearoyl phosphatidylethanolamine derivatized with polyethyleneglycol.

In another aspect, a method of treating brain cancer in a subject is provided by administering a liposome-entrapped topoisomerase inhibitor, wherein the liposome is composed of vesicle-forming lipids and having an inside/outside ion gradient effective to retain the drug within the liposome; and the topoisomerase inhibitor is selected from the group consisting of topotecan, MPE-camptothecin and CKD-602 at a concentration of at least about 0.20 μM topoisomerase inhibitor per μM lipid.

In another aspect, a method of treating brain cancer in a subject is provided by administering a liposome-entrapped topoisomerase inhibitor, wherein the liposome includes a polyanionic polymer within the liposomes, said polymer capable of forming a complex with said topoisomerase inhibitor.

In another aspect, a method of treating brain cancer in a subject is provided by administering a liposome-entrapped topoisomerase inhibitor, wherein the liposome includes a polyanionic polymer within the liposomes, said polymer capable of forming a complex with said topoisomerase inhibitor, wherein said polyanionic polymer is selected from dextran sulfate, chondroitin sulfate A, polyvinylsulfuric acid, and polyphosphoric acid.

In another aspect, a method of treating or preventing brain disorders that are associated with brain cancerin a subject is provided by administering a liposome-entrapped topoisomerese inhibitor.

European Patent No. 1,121,102 discloses many topoisomerase inhibitors and liposome-entrapped topoisomerase inhibitors. The subject matter of this patent is included in the present invention and is specifically incorporated herein by reference in its entirety.

It will be appreciated that the dose and dosing regimen can be varied to optimize the treatment of the brain cancer. The dose of the topoisomerase inhibitor can be adjusted higher or lower to achieve a desired change in the brain tumor size. Alternatively, the dosing regimen can be modified to achieve a desired decrease in the brain tumor size. For example, the dosing regimen can comprise an escalating dose for a particular period of time, followed by a constant or decreasing dose for a second period of time.

It will also be appreciated that the method can additionally include administration of a liposome-entrapped topoisomerase inhibitor in conjunction with a second therapeutic agent, in free or liposome-entrapped form. In one embodiment, a drug, such as a chemotherapeutic agent.

### EXAMPLES

The following example illustrates the method of treating brain cancer with liposome-entrapped topoisomerase inhibitors. The example is in no way intended to be limiting to the scope and spirit of the invention.

### Example 1

The objectives of the study were to evaluate the plasma, tumor, and tissue disposition of CKD-602 after a single intravenous (IV) administration of liposomes entrapped with CKD-602 and having an outer surface coating of methoxypolyethylene glycol (S-CKD602) and non-liposomal CKD-602 in female SCID mice bearing A375 human melanoma xenografts. New sample processing methods were developed to evaluate the encapsulated and released CKD-602 in plasma after administration of S-CKD602. Microdialysis methodology was used to evaluate the release of CKD-602 from S-CKD602 in tumor ECF.

Microdialysis is an in vivo sampling technique used to study the pharmacokinetics and drug metabolism in the blood and ECF of various tissues and tumor (Zamboni WC. Use of microdialysis in preclinical and clinical development. In: Handbook of Pharmacokinetics and Pharmacodynamics of Anti-Cancer Drugs, 1^{st} Ed, Figg W D, McLeod H, eds. Humana Press. 2004). The use of microdialysis methodology to evaluate the disposition of anticancer agents in tumors is relatively new. Microdialysis is based on the diffusion of non-protein-bound drugs from interstitial fluid across the semi-permeable membrane of the microdialysis probe. Microdialysis methodology allows for repeated sampling of drugs in the ECF of tissues and tumors. The released and the non-protein bound drug can be recovered due to the molecular cut off of 20 kd of the semi-permeable membrane of the microdialysis probe. Microdialysis provides a means to obtain from tumor ECF samples from which a concentration-time profile can be determined within a single tumor.

Methods

Mice. All mice were handled in accordance with the Guide to the Care and Use of Laboratory Animals (National Research Council, 1996), and studies were approved by the Institutional Animal Care and Use Committee at the University of Pittsburgh Medical Center. Mice (female C.B-17 SCID,4-6 weeks of age, and specific pathogen free), were obtained from Taconic (Hudson, N.Y.), and were allowed to acclimate to the animal facilities at the University of Pittsburgh for 1 week prior to initiation of study. Mice were housed in microisolator cages and allowed ISDPRO autoclavable rodent chow (PMI Nutrition International, Inc., Brentwood, Mo.) and mice received water ad libitum. Body weights and tumor size were measured twice weekly and clinical observations were made twice daily.

Tumor Lines. A375 human melanoma xenografts were obtained from the DCTD Tumor Repository (Fredrick, Md.) and were mouse antigen production test-negative. A375 tumors were expanded in culture and injected (1×10⁷ cells/mouse) subcutaneously into passage mice. The A375 tumors were harvested when they were 1 to 2 g and were implanted as approximately 25-mg fragments subcutaneously on the fight flank of SCID mice by aseptic techniques. Tumor volumes were calculated from the formula: length×(width)²/2, where length is the largest diameter and width is the smallest diameter perpendicular to the length (Zamboni et al., Relationship between systemic exposure of 9-nitrocamptothecin and its 9-aminocamptothecin metabolite and tumor response in human colon tumor xenografts. Clin Cancer Res 11(13):4867-74:2005). Pharmacokinetic and microdialysis studies were performed when the tumors were approximately 1000 to 1500 mm³ (1 to 1.5 g) in size.

Formulation and Administration. S-CKD602 is a pegylated liposomal formulation of CKD-602. The clinical formulation of S-CKD602 was used in this study (Alza Corp., Mountain View Calif.) (Zamboni et al., Final results of a phase I and pharmacokinetic study of STEALTH liposomal CKD-602 (S-CKD602) in subjects with advanced solid tumors. Proceedings of ASCO 24(2013);82s:2006). Approximately 80% of the lipid in S-CKD602 liposome is fully hydrogenated soy phosphatidylcholine and cholesterol. In addition, methoxypolyethylene glycol is covalently bound to phosphatidylethanolamine and a component of the lipid bilayer. The mean particle size of the S-CKD602 was approximately 110 nm. CKD-602 is encapsulated in the core of the liposome with an encapsulation efficiency of greater than 90%. The drug-to-lipid ratio of S-CKD602 is approximately 14 g CKD-602 per milligram of lipid. In the S-CKD602 formulation, the CKD-602 concentration was 0.1 mg/mL. The doses of S-CKD602 refer to actual doses of CKD-602. S-CKD602 was administered at 1 mg/kg IV push via a tail vein over approximately 1 min. This dose is one-half the maximum tolerated dose (MTD) in mice. The dose of S-CKD602 administered was based on the maximum volume of drug allowed to be administered IV by the study's IACUC.

Non-liposomal CKD602 was administered at 30 mg/kg IV push via the tail vein. This dose is approximately the MTD for a single dose of non-liposomal CKD-602 in mice. Non-liposomal CKD-602 was prepared at 3 mg/mL in 270 mM mannitol and 0.4 mM tartaric acid in a 5% dextrose solution at pH 3.6. The vehicle control for S-CKD602 and non-liposomal CKD-602 was 0.9% NaCI and 270 mM mannitol and 0.4 mM tartaric acid in a 5% dextrose solution at pH 3.6, respectively.

Pharmacokinetic Studies. Due to limited sample volume, the pharmacokinetic and microdialysis studies were performed in separate groups of mice. Pharmacokinetic studies of S-CKD602 were performed after administration of vehicle, and at 5 min, 0.25, 0.5, 1, 2, 4, 7, 16, 24, 48, and 72 h after administration. Pharmacokinetic studies of non-liposomal CKD-602 were performed after administration o f vehicle, and at 5 min, 0.25, 0.5, 1, 2, 4, 6, 16, and 24 h after administration. For each pharmacokinetic study, mice (n=3 per time point) were euthanized with carbon dioxide and heparinized blood samples (approximately 0.8 to 1 mL) were collected by cardiac puncture. The blood samples were centrifuged at 12,000× g for 4 min. After S-CKD602 administration, the plasma was processed to measure encapsulated, released, and sum total (encapsulated+released) CKD-602. For S-CKD602 and non-liposomal CKD-602, tumor, liver, kidney, spleen, brain, peritoneal cavity fat, and bicep femoris skeletal muscle samples were obtained for measurement of sum total drug.

Sample Processing. The plasma for the pharmacokinetic studies of S-CKD602 was immediately placed on ice but not frozen. Plasma samples of S-CKD602 can not be frozen because freezing the plasma sample ruptures the liposome and thus prevents the differentiation between encapsulated and released drug. After administration of S-CKD602, sum total, encapsulated, and released CKD-602 concentrations in plasma were measured in separate aliquots from the same sample. The separation of liposomal and encapsulated and released CKD-602 was accomplished by solid phase separation (SPS). An aliquot of 100 μL plasma was spiked with internal standard (I.S.)(10 μL of D7-CKD-602, 5 ug/mL) was loaded onto a BondElute LRC SPE cartridge (Varian, Harbor City, Calif.). The cartridge was preconditoned with 2 mL methanol and 2 mL 0.9% saline. The encapsulated CKD-602 was eluted and collected with 2 mL of 0.9% saline. The cartridge was then washed with an additional 5 mL of 0.9% saline to remove any remaining encapsulated CKD-602 and 2 mL water to remove salt from the cartridge. Released CKD-602 and internal standard were eluted with 1 mL acetonitrile acidified with 0.1% formic acid.

The encapsulated CKD-602 samples in plasma were processed by taking a 200 μL aliquot of the 0.9% saline elutant and adding 10 μL of internal standard. Salt was removed with 200 μL methylene chloride and 200 μL 50 mM ammonium acetate, pH 8.3. Samples were centrifuged at 20,000 RCF for 6 min at 5° C. The organic layer, (bottom layer) was transferred to 10×75 mm borosilicate glass tubes and dried under nitrogen gas at 37° C. The dried residue was suspended in 100 μL methanol:water (35:65, v/v) containing 0.1% formic acid, transferred into autosampler vials, and centrifuged at 10,000 RCF for 6 min at 5° C. to remove the particulates.

The sample processing for released CKD-602 in plasma was performed by taking 1 mL of acidified acetonitrile elute and 10 μL internal standard. The samples were vortexed and then centrifuged at 20,000 RCF for 6 min at 5° C. The supernatants were decanted into 10×75 mm borosilicate glass tubes and dried under nitrogen gas at 37° C. The dried residue was suspended in 100 μL methanol:water (35:65, v/v) containing 0.1% formic acid. The sample processing of plasma for sum total CKD-602 was performed using the addition of 1 mL of acetonitrile as described above for released CKD-602.

Plasma samples for non-liposomal CKD-602 were immediately frozen in liquid nitrogen, and stored at −80° C. until analyzed. The sample processing of plasma for CKD-602 was performed using the addition of 1 mL of aceonitrile as described above for released CKD-602. Tumor and tissue samples for S-CKD602 and non-liposomal CKD-602 were weighed, snap frozen in liquid nitrogen, and stored at −80° C. until analyzed. The sum total sample of CKD-602 in tumor and tissues were processed by homogenizing tissues in PBS, pH 7.0, at 1:3 (w/v). Aliquots of 100 μL homogenate were then transferred to microcentrifuge tubes, spiked with 10 μL internal standard, and extracted with 500 μL acidified acetonitrile. The samples were centrifuged at 20,000 RCF for 6 min at 5° C. The supernatants were decanted into 10×75 mm borosilicate glass tubes and dried under nitrogen gas at 37° C. The dried residue was suspended in 100 μL methanol:water (35:65, v/v) containing 0.1% formic acid.

Microdialysis Studies of Tumor ECF Disposition. Microdialysis studies were performed to evaluate the tumor ECF disposition of non-liposomal CKD-602 and released CKD-602 from S-CKD602. After administration of S-CKD602, microdialysis studies (n=3 to 4 mice per interval) were obtained every 20 min from 0 to 2 h and every 30 min from 24 to 27 h, 48 to 51 h, and 72 to 75 h. After administration of non-liposomal CKD-602, microdialysis studies (n=3 to 4 mice per interval) were obtained every 20 min, from 0 to 2 h, and every 30 min, from 4 to 7 h and 20 to 24 h. Microdialysis probe recovery was estimated using retrodialysis calibration from 0 to 2 h after administration of S-CKD602 and non-liposomal CKD-602 as previously described. At all other microdialysis sample intervals, probe recovery was estimated using camptothecin as a tracer agent. Tumor ECF samples of CKD-602 after administration of S-CKD602 and non-liposomal CKD-602 were processed by adding 10 μL of the I.S. At the end of each microdialysis procedure, plasma was processed to measure encapsulated, released, and sum total S-CKD602. Tumor and tissue samples were also obtained and processed as described above to measure sum total CKD-602.

Analytical Studies. An LC/MS assay was used to measure the camptothecin total (sum of lactone and hydroxy acid) forms of encapsulated, released, and sum total CKD-602 in plasma, sum total CKD-602 in tumor and tissues, and CKD-602 in tumor ECF after administration of S-CKD602. In addition, the LC/MS assay was used to measure CKD-602 in plasma, tumor, and tissues after administration of non-liposomal CKD-602. This LC/MS assay was modified from a previous assay for 9-nitrocamptothecin (See Zamboni et al., Relationship between systemic exposure of 9-nitrocamptothecin and its 9-aminocamptothecin metabolite and tumor response in human colon tumor xenografts. Clin Cancer Res 11 (13):4867-74:2005).

The HPLC system consisted of a Finnigan Specta Systems AS3000 autosampler and P4000 quarternary pump (Thermo Finnigan, Waltham, Mass.) with a Phenomenex Synergi Hydro-RP 80A (4 um, 100×2 mm) analytical column (Phenomenex, Torrance, Calif.). The isocratic mobile phase consisted of 0.1% formic acid in methanol:water (35:85, v/v) and was pumped at 0.2 mL/min. Injection volume was 10 pL and the run time was 10 min. Column eluant was analyzed with a ThermoFinnigan aQa mass spectrometer (Thermo Quest, San Jose, Calif.) operating in electrospray positive mode electron ionization monitoring CKD-602 and the I.S. D7-CKD-602 at 434.1 m/z and 441.2 m/z, respectively. The insert probe temperature was set at 400° C. with 3 kV ion spray voltage and 20 V orifice voltage. Nitrogen gas flow was fixed at 75 p.s.i. at the tank head unit. The system was operated with ThermoFinnigan Xcaliber software. The CKD-602 to I.S. ratio was calculated for standards by dividing the analyte peak area by that of the I.S. Standard curves for CKD-602 were constructed by plotting the analyte to I.S. ratio versus the known concentration of the analyte in each standard. Standard curves were fit by linear regression with 1/y² weighting and back calculation of CKD-602 concentrations.

Results

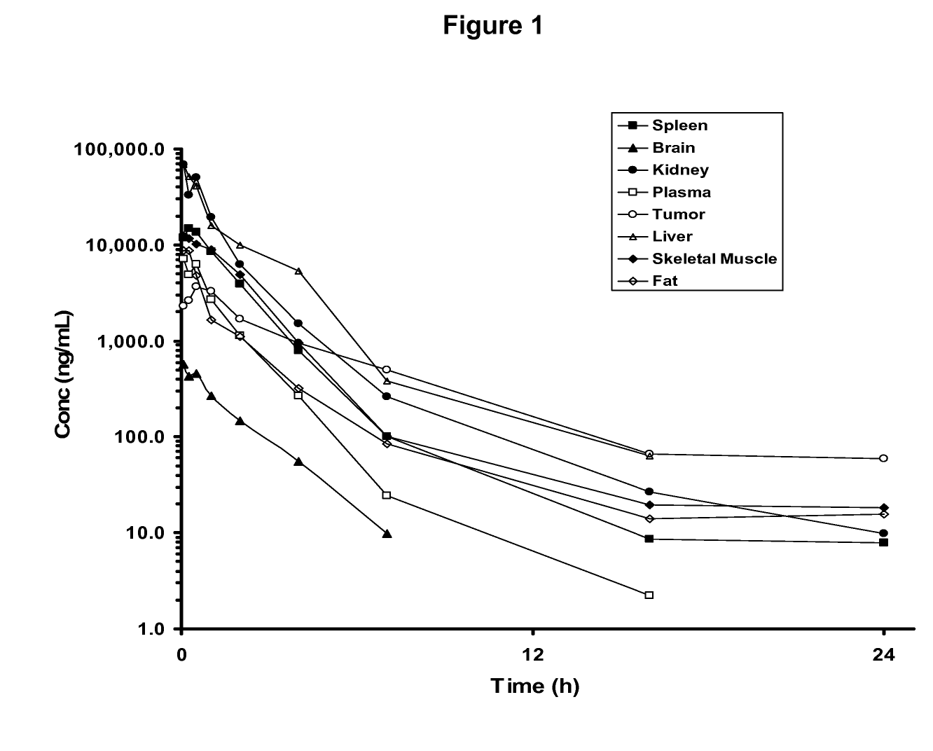

Plasma, Tissue and Tumor Sum Total Pharmacokinetic Disposition. The plasma, tissue, and tumor pharmacokinetic disposition of sum total CKD-602 was compared after administration of non-liposomal CKD-602 and S-CKD602. The concentration versus time profile of sum total CKD-602 in plasma, tissue and tumors after administration of non-liposomal CKD-602 is presented in FIG. 1. The sum total pharmacokinetic parameters after administration of non-liposomal CKD-602 are presented in Table 1. After administration of non-liposomal CKD-602, the plasma concentration versus time profile of CKD-602 peaked at 0.083 h (5 min) after administration, had a bi-phasic elimination profile, and was no longer detectable after 16 h. The concentration versus time profiles of CKD-602 in all tissues were similar to the profile in plasma. The exposure of CKD-602 was higher in tumor compared with plasma and the other tissues from 7 to 24 h. Consistent with the distribution and elimination of other non-liposomal camptothecin analogues, the highest exposures of CKD-602 in tissues after administration of non-liposomal CKD-602 were in the liver and kidney. The overall distribution of non-liposomal CKD-602 was 3-fold greater in muscle compared with fat.

TABLE 1
Sum Total Pharmacokinetic Parameters After Administration of
S-CKD602 and Non-Liposomal CKD-602 in Mice Bearing A375 Tumors

| Parameter | | | | | | | | |
|---|---|---|---|---|---|---|---|---|
| (units) | Plasma | Tumor | Liver | Kidney | Spleen | Fat | Muscle | Brain |
| S-CKD602 | | | | | | | | |
| AUC (ng/mL · h) | 201,929 | 13,194 | 39,667 | 18,919 | 42,294 | 4,321 | 3,399 | 2,170 |
| Cₘₐₓ (ng/mL) | 18,246 | 280 | 2090 | 707 | 1,657 | 281 | 126 | 205 |
| Tₘₐₓ (h) | 0.08 | 7.00 | 0.083 | 0.5 | 0.5 | 0.08 | 1 | 0.5 |
| Tₗₐₛₜ (h) | 72 | 72 | 48 | 72 | 72 | 72 | 72 | 48 |
| Non-Liposomal | | | | | | | | |
| CKD-602 | | | | | | | | |
| AUC (ng/mL · h) | 9,117 | 11,661 | 68,620 | 56,267 | 23,124 | 8,280 | 24,485 | 849 |
| Cₘₐₓ (ng/mL) | 6,959 | 3,688 | 69,000 | 68,497 | 14,724 | 7,321 | 12,383 | 572 |
| Tₘₐₓ (h) | 0.08 | 0.50 | 4 | 0.08 | 0.25 | 0.25 | 0.08 | 0.08 |
| Tₗₐₛₜ (h) | 16 | 24 | 16 | 24 | 24 | 24 | 24 | 7 |

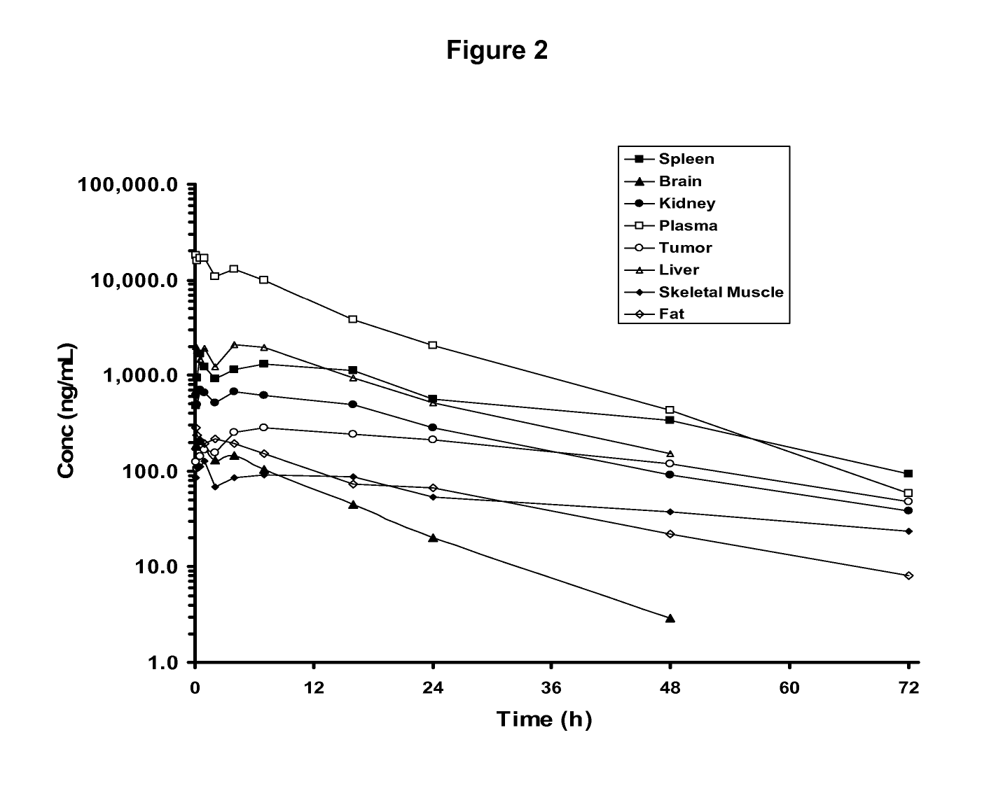

The concentration versus time profile of sum total CKD-602 in plasma, tissue and tumors after administration of S-CKD602 is presented in FIG. 2. The sum total pharmacokinetic parameters after administration of S-CKD602 are presented in Table 1. After administration of S-CKD602, the plasma concentration versus time profile of CKD-602 peaked at 0.083 h (5 min) after administration, was maintained for approximately 4 h, and then had a single phase elimination profile, and was detectable at 72 h after administration. The concentration versus time profiles of sum total CKD-602 in all other tissues were similar to the profile in plasma. Consistent with the tissue distribution of liposomal encapsulated drugs, the highest exposures of sum total CKD-602 in tissues after administration of S-CKD602 were in the spleen, tumor, and liver. The distribution of S-CKD602 from 0 to 24 h was 1.6-fold greater to fat compared with muscle. In addition, the overall distribution of S-CKD602 was 1.3-fold greater to fat as compared with muscle. The ratio of CKD-602 sum total exposure in fat to muscle was 3.8-fold higher after administration of S-CKD602 compared with non-liposomal CKD-602.

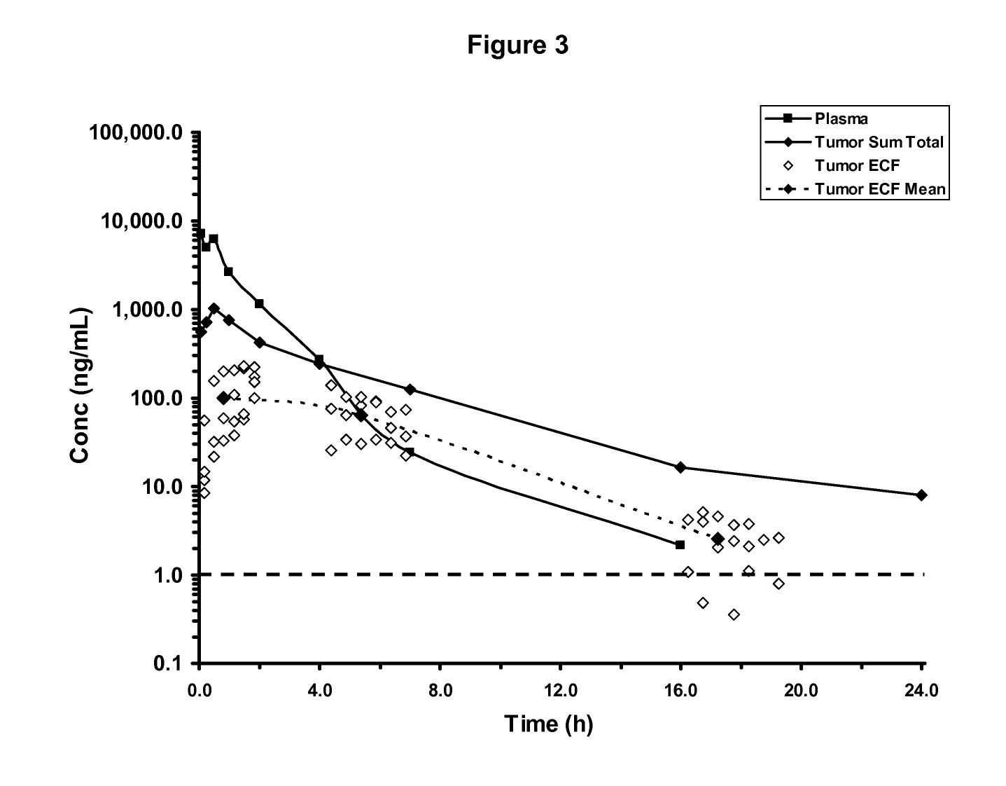

Plasma and Tumor Disposition of Encapsulated and Released CKD-602. The plasma, tumor, and tumor ECF pharmacokinetic disposition of CKD-602 was compared after administration of non-liposomal CKD-602 and S-CKD602. The plasma, tumor, and tumor ECF disposition of CKD-602 after administration of non-liposomal CKD-602 is presented in FIG. 3 and below in Table 2.

TABLE 2
Plasma, Tumor, and Tumor ECF Pharmacokinetic Parameters
for S-CKD602 and Non-liposomal CKD-602 in Mice Bearing A375
Human
Melanoma Xenografts

| | | | Non- |
|---|---|---|---|
| | | S-CKD602 | Liposomal CKD-602 |
| Parameters | Units | (1 mg/kg) | (30 mg/kg) |
| Plasma | | | |
| Sum Total AUC_{(0-inf)} | ng/mL · h | 201,929 | 9,117 |
| Encap AUC_{(0-inf)} | ng/mL · h | 165,717 | — |
| Released AUC_{(0-inf)} | ng/mL · h | 36,905 | — |
| Tumor | | | |
| Tumor Sum Total_{(0-inf)} | ng/mL · h | 13,194 | 11,661 |
| Tumor ECF AUC_{(0-inf)} | ng/mL · h | 187 | 639 |
| Time >1 ng/mL In | h | >72 | ˜20 |
| Tumor ECF | | | |

The concentrations of sum total CKD-602 were higher in plasma compared to tumor from 0.083 h (5 min) to 2 h and then were higher in tumor compared to plasma from 7 h to 24 h. The concentration versus time profile of CKD-602 in tumor ECF were detectable from 10 min to 19.25 h and were consistent with the profile of sum total CKD-602 in tumor homogenates. In addition, the concentration of CKD-602 in tumor ECF varied 4- to 5-fold at individual time points during the collection intervals of 0 to 2 h, 4 to 8 h, and 16 to 20 h. The difference in the CKD-602 measured in samples obtained from tumor homogenate (11,661 ng/mL•h) and tumor ECF (639 ng/mL•h) may be due to binding of CKD-602 to plasma proteins or proteins within the tumor matrix.

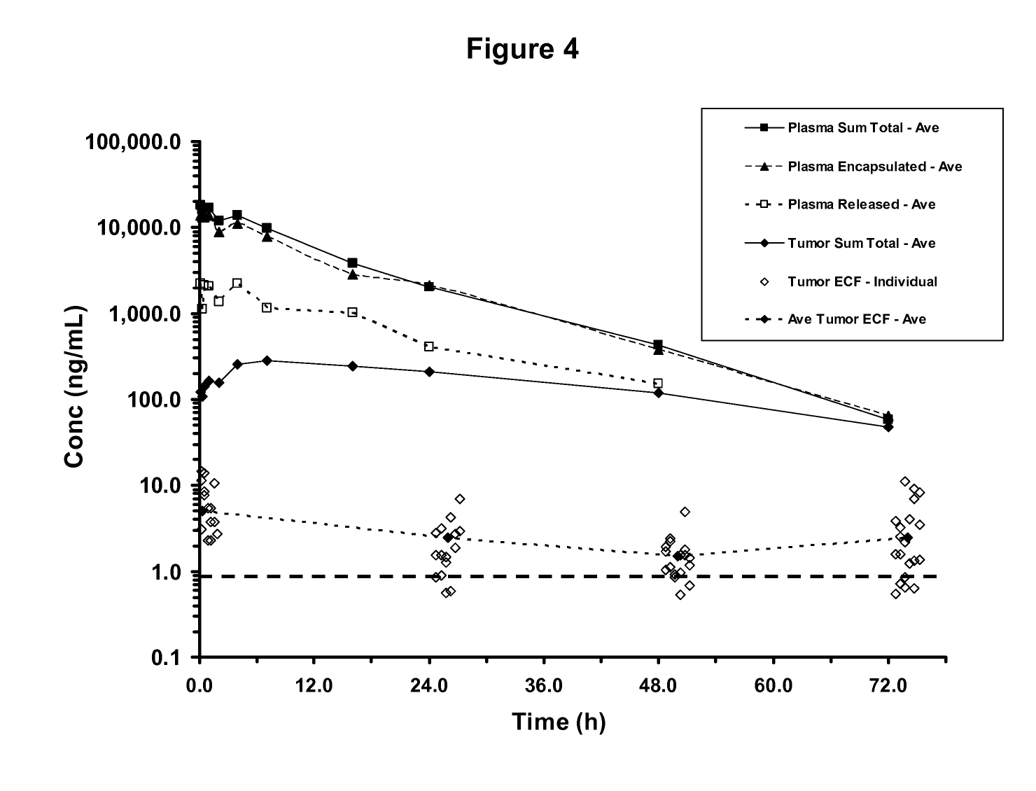

The plasma, tumor, and tumor ECF disposition of CKD-602 after administration of S-CKD602 is presented in FIG. 4 and Table 2. The concentration of sum total and encapsulated CKD-602 were detectable from 5 min to 72 h and the released CKD-602 was detectable from 5 min to 48 h after administration of S-CKD602. The concentration versus time profile of released CKD-602 was similar to the profiles of sum total and encapsulated CKD-602, and ratio of released CKD-602 to sum total or encapsulated CKD-602 was consistent suggesting that the release of CKD-602 from the liposome is constant. Approximately 82% of CKD-602 remains encapsulated in plasma, as estimated by the ratio of released CKD-602 AUC to sum total CKD-602 AUC or the difference between sum total CKD-602 AUC and encapsulated CKD-602 AUC.

After administration of S-CKD602, the concentration versus time profile of sum total CKD-602 measured in tumor homogenates peaked at 7 h and remained relatively constant from 7 h to 48 h. The concentration versus time profile of CKD-602 in tumor ECF were detectable from 10 min to 75.25 h after administration of S-CKD602 which is significantly greater than after administration of non-liposomal CKD-602. The concentration versus time profile of CKD-602 in tumor ECF was consistent with the profile of sum total CKD-602 in tumor homogenates. In addition, the concentration of CKD-602 in tumor ECF varied approximately 10-fold at individual time points during the each of the collection intervals. The difference in the CKD-602 measured in samples obtained from tumor homogenate (13,194 ng/mL•h) and tumor ECF (187 ng/mL•h) most likely due to the slow release of CKD-602 from the liposome and binding of CKD-602 to plasma proteins or proteins within the tumor matrix because the tumor ECF samples were obtained using microdialysis methodology which can only recovery released non-protein (albumin) bound drug due to the molecular weight cut off the probe.

Discussion

This was the first study reporting prolonged exposure of released drug in plasma and tumor ECF after administration of a liposomal anticancer agent; however, this was the first study reporting prolonged exposure of released drug in tumor ECF after administration of a liposomal anticancer agent having an outer surface coating of hydrophilic polymer chains. In addition, this was the first study evaluating the distribution of a liposomal agent having an outer surface coating of hydrophilic polymer chains compared with a non-liposomal drug to fat and muscle. The results of this study strongly suggested that S-CKD602 provides pharmacokinetic advantages in plasma and tumors when compared to the non-liposomal formulation of CKD-602 at 1/30^{th} the dose. In addition, the results of the study were consistent with the improved antitumor efficacy and therapeutic index of S-CKD602 compared with non-liposomal CKD-602.

The ideal pharmacologic characteristics of a liposomal, nanoparticle, or conjugated anticancer agent are prolonged circulation of the encapsulated drug in the blood or plasma, high tumor delivery, and the release of drug from the carrier into the tumor ECF. S-CKD602 meets all of these pharmacologic criteria. The sum total plasma exposure of S-CKD602 was approximately 25-fold greater than non-liposomal CKD-602. After administration of S-CKD602, 82% of the CKD-602 remained encapsulated in plasma. The overall tumor delivery, as measured by the exposure of CKD-602 in tumor homogenates, were similar after administration of S-CKD602 and non-liposomal CKD-602; however, the duration of exposure was approximately 3-fold longer for S-CKD602 compared with non-liposomal CKD-602. Moreover, the time the concentrations of CKD-602 were >1 ng/mL in the tumor ECF was at least 3.6-fold longer after S-CKD602 compared with non-liposomal CKD-602. The importance of detecting released drug in the tumor ECF after administration of a liposomal anticancer agent is that the encapsulated drug can not penetrate into the cell and thus it is an inactive-prodrug and that only the released-drug can penetrate into the cell and thus is active. The importance of the duration of time the concentrations exceeds 1 ng/mL is based on studies evaluating the threshold concentration associated with in vitro cytotoxicity for other camptothecins analogues. These results are consistent with the antitumor response to camptothecin analogues which is related to the duration of exposure of cytotoxic concentrations.

After administration of non-liposomal CKD-602, the highest exposure was in the liver and kidney, which is consistent with the clearance of camptothecin analogues (Stewart et al., Topoisomerase I interactive drugs in children with cancer. Investigational New Drugs 14:37-47; 1996; Sparreboom et al., Topoisomerase I Inhibitors. In Chabner B A and Longo D L, editors. Cancer Chemotherapy and Biotherapy: Principles and Practice, Fourth Edition, Lippincott Williams & Wilkins, 2005). After administration of S-CKD602, the highest exposures were in the spleen and liver. The high exposure of drug in spleen after administration of S-CKD602 is consistent with previous studies of conventional and pegylated liposomal drugs and is believed to be due to the presence of the reticuloendothelial system (RES) in these tissues. Thus, the mPEG-coating on liposomes does not prevent the clearance via the RES but slows the clearance of the pegylated drugs via the RES compared with conventional or non-pegylated liposomes. The only way to fully evaluate the extent by which the RES clears pegylated versus non-pegylated liposomes is to evaluate the plasma, spleen, and liver disposition of each type of liposomal formulation; however non-pegylated liposomal formulation of CKD-602 has not been developed. As the activity of the RES may be a factor that affects delivery and release of drug from liposomes in plasma and tissue, it may also affect the delivery of liposomal drugs to tumors. However, these factors are currently unclear and further exploration of the RES function and activity as related to the disposition of liposomal anticancer agents in tissues and tumors needs to be evaluated.

Previous studies evaluating the pharmacokinetics and tissue distribution of a liposomal encapsulated drug have not evaluated the disposition of drug in fat and muscle. The distribution of non-liposomal CKD-602 was greater to muscle compared with fat, whereas the distribution of S-CKD602 was greater to fat compared with muscle. Since approximately 18% of the CKD-602 is released into plasma after administration of S-CKD602 some of the exposure in muscle after administration of S-CKD602 may be associated with the released drug relative to the encapsulated CKD-602. Thus, the results of the study may be an under estimate of the difference in the distribution of CKD-602 to fat and muscle after administration of S-CKD602. These results suggest that the body composition and catabolism of S-CKD602 in subjects may affect the disposition of S-CKD602 and released CKD-602.

The brain exposure of CKD-602 was 2.4-fold higher after administration of S-CKD602 compared with non-liposomal CKD-602. This increased penetration of drug after administration of hydrophilic polymer-coated liposomal drugs has also been reported for hydrophilic polymer-coated liposomal formulations of doxorubicin (Sharma et al., Liposomal-mediate therapy of intracranial brain tumors in a rat model. Pharm Res 14(8):992-8:1997; Zou et al., Organ distribution and tumor uptake of annamycin, a new anthracycline derivative with high affinity for lipid membranes, entrapped in multilamellar vesicles. Cancer Chemother Pharmacol 32(3);190-6:1993). The mechanism by which liposomal agents penetrate into the brain is unknown. The greater exposure of CKD-602 in the brain after S-CKD602 compared with non-liposomal CKD602 and activity of camptothecin analogues in subjects with brain tumors suggest S-CKD602 as a therapeutic in this subject population. It was unexpected that administration of S-CKD602 led to the passage of CKD-602 pass the blood brain barrier and accumulation of CKD-602 in brain tissue that has been compromised by cancer, as well as brain tissue that has not been compromised by cancer.

The development of liposomes having an outer surface coating of hydrophilic polymer chains was based on the discovery that incorporation of PEG-lipids into liposomes yields preparations with prolonged plasma exposure, superior tumor delivery, and increased antitumor effect compared with the non-liposomal formulation of the drug. S-CKD602 has all of these pharmacologic and cytotoxic advantages. In addition, these advantages are associated with administration of a single IV dose of S-CKD602, whereas as with other camptothecin analogues the non-liposomal formulation of CKD-602 needs to be administered for several consecutive days in order to maintain drug exposure threshold and achieve antitumor activity. Thus, S-CKD602 also has clinical and logistical advantageous over topotecan, which is administered IV daily for 5 days repeated every 21 days in the treatment of ovarian cancer and small cell lung cancer.

Although the invention has been described with respect to particular embodiments, it will be apparent to those skilled in the art that various changes and modifications can be made without departing from the invention.

What is claimed is:

1. A method of treating brain cancer, the method comprising administering to a subject a liposome comprising at least one topoisomerase inhibitor entrapped in said liposome.

2. The method of claim 1, wherein the liposome has an outer surface coating of hydrophilic polymer.

3. The method of claim 1, wherein the liposome is composed of at least about 20 mole percent of a vesicle-forming lipid and at least about 1 mole percent of a vesicle-forming lipid derivatized with a hydrophilic polymer, said polymer being distributed on both sides of the liposomes' bilayer membrane.

4. The method of claim 1, wherein entrapped in the liposome is a topoisomerase inhibitor at a concentration of at least about 0.10 μM drug per μM lipid.

5. The method of claim 1, wherein the liposome has an inside/outside ion gradient sufficient to retain the topoisomerase inhibitor within the liposome at a specified concentration.

6. The method of claim 1, wherein the topoisomerase inhibitor is a topoisomerase I inhibitor selected from the group consisting of camptothecin and camptothecin derivatives.

7. The method of claim 6, wherein the camptothecin derivative is selected from the group consisting of 9-aminocamptothecin, 7-ethylcam ptothecin, 10-hydroxycamptothecin, 9-nitrocamptothecin, 10,11-methylenedioxycamptothecin, 9-amino-10,11-methylenedioxycamptothecin, and 9-chloro-10,11-methylenedioxycamptothecin.

8. The method of claim 6, wherein the camptothecin derivative is selected from the group consisting of irinotecan, topotecan, (7-(4-methylpiperazinomethylene)-10,11-ethylenedioxy-20(S)-camptothecin, 7-(4-methylpiperazinomethylene)-10,11-methylenedioxy-20(S)-camptothecin and 7-(2-N-isopropylamino)ethyl)-(20S)-camptothecin.

9. The method of claim 1, wherein the topoisomerase inhibitor is a topoisomerase I/II inhibitor selected from the group consisting of 6-[[2-(dimethylamino)-ethyl]amino]-3-hydroxy-7H-indeno[2,1-c]quinolin-7-one dihydrochloride, azotoxin and 3-methoxy-11H-pyrido[3′,4′-4,5]pyrrolo[3,2-c]quinoline-1,4-dione.

10. The method of claim 1, wherein the topoisomerase inhibitor is CKD-602.

11. The method of claim 2, wherein the hydrophilic polymer is polyethyleneglycol having a molecular weight between 500-5,000 daltons.

12. The method of claim 1, wherein the brain cancer is a primary brain tumor.

13. The method of claim 12, wherein the brain cancer is a cancerous primary brain tumor.

14. The method of claim 12, wherein the brain cancer is a glioma priary brain cancer.

15. The method of claim 1, wherein the brain cancer is a metastatic brain cancer.
